(19) United States
(12) Patent Application Publication
(10) Pub. No.: US 2009/0081090 A1
(43) Pub. Date: Mar. 26, 2009

(54) Sterilizer with modular pressure housing for a sterilization chamber

(76) Inventors: Jeffrey G. Dayman, Waterloo (CA); Christopher John Howard Stewart, Toronto (CA)

Correspondence Address: CLARK & BRODY, 1090 VERMONT AVENUE, NW, SUITE 250, WASHINGTON, DC, 20005, US

(21) Appl. No.: 11/902,814

(22) Filed: Sep. 26, 2007

(51) Int. Cl. A61L 2/07; A61L 2/26

(52) U.S. Cl. 422/297

(57) ABSTRACT

There is provided an apparatus for sterilizing medical or dental instruments. The apparatus comprises a sterilizing unit, a cassette for holding the instruments within the unit during sterilization and pressure housing removably attached to the sterilizing unit to receive the cassette during use. The pressure housing is configured to be releasably connectable to the sterilizing unit to permit the efficient coupling and de-coupling of the pressure housing to steam delivery means, exhaust means and control means of the sterilizing unit.

### FIELD OF THE INVENTION

This invention relates to an apparatus for the sterilization of medical and dental instruments using a cassette housed within a modular pressure housing releasably connected to a sterilizing unit.

### BACKGROUND OF THE INVENTION

The cassette autoclave has represented a significant advancement in the art of steam sterilization of medical and dental instruments. In particular, the cassette autoclave has been widely used as a simple but elegant solution to the sterilization of dental instruments in a dentist's office. It has provided a compact apparatus to sterilize dental instruments that is easy to operate and highly effective at sterilization. It has also been widely used in surgical environments and other applications where sterile instruments are required, such as laser eye surgery clinics.

An example of such a cassette autoclave is the applicant's STATIM™ unit, embodied in several patents worldwide, including U.S. Pat. No. 5,271,893 of Newman.

The conventional cassette autoclaves have a pressure housing to provide a compartment for the cassette within the sterilizing unit during operation. The cassette is typically thin walled and lightweight. It must, nonetheless withstand significant stresses during pressurization in operation of the unit. The pressure housing thus functions to provide structural integrity to the cassette during operation. Therefore, the pressure housing is typically constructed of cold-rolled steel, a combination of cold and hot rolled steel or similar materials. The pressure housing is thus relatively heavy and can often account for up to 50% of the weight of the entire autoclave.

The weight of the pressure housing makes it difficult to maneuver and service the autoclave. Transportation of the autoclave to and from a service depot can be quite costly due to its weight.

At present, the pressure housing is designed to have the inlet and exhaust ports permanently connected to the pressure housing within the autoclave. This is in part to ensure that the ports as well as the temperature sensor protruding from the exhaust port of the autoclave mate accurately with the cassette when the cassette is in place within the pressure housing. The tolerances of aligning the inlet and exhaust ports with the cassette are extremely tight, especially considering the possibility and therefore any movement of interchanging different cassettes within a single autoclave. Misalignment of the ports could result in a malfunctioning unit and potential damage to the temperature sensor, the cassette or both. The permanent mechanical and electrical connections of the ports and temperature sensor to the pressure housing make it difficult, if not impossible, to separate the pressure housing from the rest of the autoclave.

However, improvements in the use of probes to house the temperature sensor and self-aligning couplings in the cassette, as described in applicant's co-pending PCT application Publication No. WO 00/59552 have eased the tolerances in placing the temperature sensor within the cassette. Thus, so long as the positions of the ports are controlled, the temperature sensor will be properly inserted into the cassette.

An autoclave in which the pressure housing can be easily removed and re-installed within the autoclave would thus represent several significant improvements.

### SUMMARY OF THE INVENTION

Embodiments of the present invention provide an apparatus for sterilizing, and in certain embodiments, drying of medical and dental instruments using steam.

In accordance with an embodiment of the invention, there is provided an apparatus for sterilizing medical or dental instruments. The apparatus comprises a sterilizing unit, a cassette for holding the instruments within the unit during sterilization and a pressure housing removably attached to the sterilizing unit to form a compartment to receive the cassette during use. The pressure housing releasably connects to the sterilizing unit to allow the ingress and egress of steam into the cassette during sterilization of the instruments.

In another embodiment of the invention there is provided an apparatus for sterilizing medical or dental instruments. The apparatus comprises a sterilizing unit housing steam delivery means, exhaust means and control means for the apparatus, a cassette for holding the instruments within the unit during sterilization and a pressure housing forming a compartment to receive the cassette during use. The pressure housing is configured to be releasably connectable to the sterilizing unit to permit the efficient coupling and de-coupling of the pressure housing to the steam delivery means, exhaust means and control means.

### BRIEF DESCRIPTION OF THE DRAWINGS

These and other advantages of the invention will become apparent upon reading the following detailed description and upon referring to the drawings in which:

FIG. 1. is a perspective view of an apparatus for sterilizing medical and dental instruments of the type contemplated for use with the present invention.

FIG. 2. is a perspective view of the apparatus of FIG. 1 with the cover, water reservoir and cassette removed.

FIG. 3. is a perspective view, partially cutaway, of the pressure housing of the present invention, positioned within the apparatus of FIG. 1.

FIG. 3A is a detail view of a section of FIG. 3.

FIG. 4. is a perspective view of the pressure housing of the present invention, partially removed from the apparatus.

FIG. 5. is a perspective view of the chassis of the apparatus.

FIG. 6 is a rear perspective view of the pressure housing of the present invention.

FIG. 7. is a perspective view, partially cutaway, of the pressure housing of the present invention positioned within the apparatus of FIG. 1.

FIG. 7A is a detail view of a section of FIG. 7.

FIG. 8. is a perspective view, partially cutaway, of the pressure housing of the present invention positioned within the apparatus of FIG. 1, with the cassette installed.

FIG. 8A is a detail view of a section of FIG. 8.

FIG. 9 is a perspective view of the pressure housing of the present invention.

FIG. 10 is a cutaway view of the pressure housing of the present invention.

While the invention will be described in conjunction with the illustrated embodiments, it will be understood that it is not intended to limit the invention to such embodiments. On the contrary, it is intended to cover all alternatives, modifications, and equivalents as may be included within the spirit and scope of the invention as defined by the appended claims.

### DETAILED DESCRIPTION OF THE PREFERRED EMBODIMENTS

In the following detailed description, similar features in the drawings have been given identical reference numerals where appropriate.

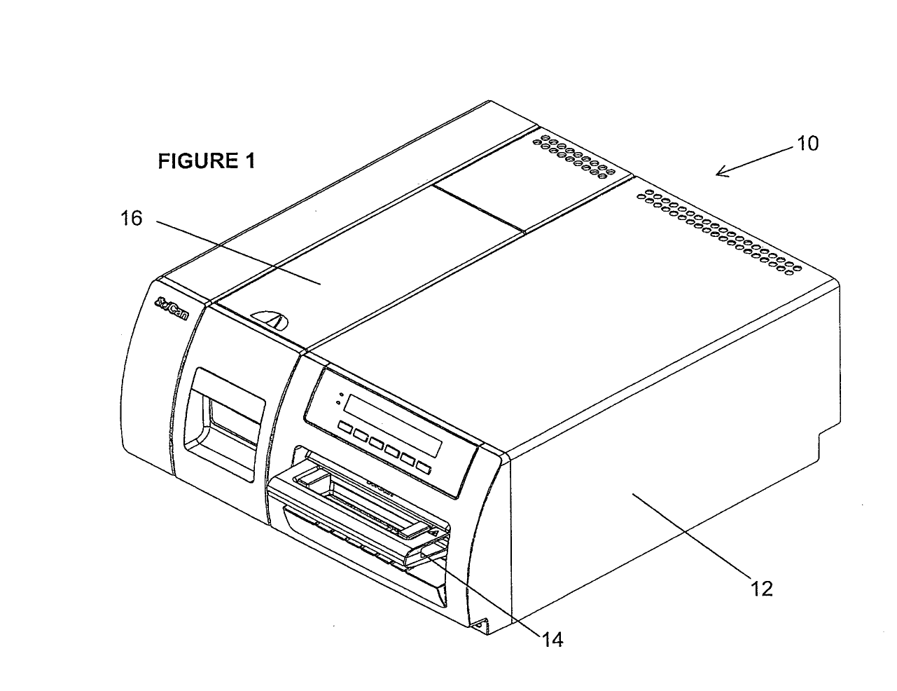

FIG. 1 illustrates an apparatus 10 for sterilizing medical and dental instruments (not shown). The apparatus 10 is shown for illustration purposes as a cassette autoclave, such as that manufactured and sold by the applicant under the trade-mark STATIM. It is contemplated, however, that the present invention may be suitable for use in other cassette or chamber type sterilizers.

As seen in FIG. 1, the apparatus in the illustrated example, comprises a cover assembly 12, a cassette assembly 14 and a reservoir assembly 16. The cassette assembly 14 is shown inserted into the apparatus 10, in the operable position, in which the cassette 14 would hold the instruments to be sterilized.

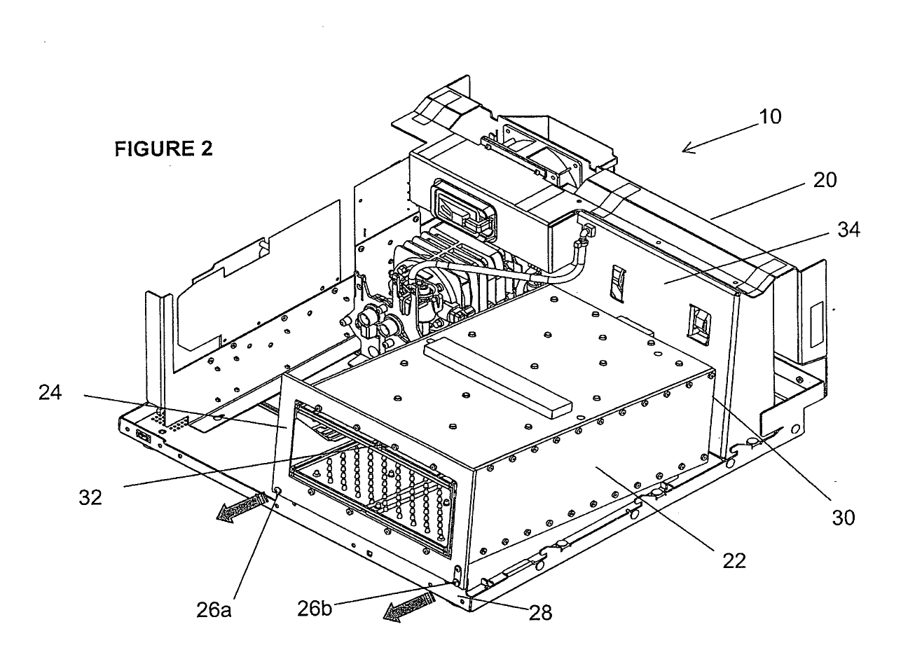

FIG. 2 illustrates the apparatus 10 with the cover assembly 12, the cassette 14 and the reservoir assembly 16 removed, thus revealing the chassis 20 of the apparatus 10 and the pressure housing 22. The chassis 20 contains the serviceable module of the apparatus 10, including the water supply interface, the electronic control systems and the steam inlets and exhausts. Although the design of the serviceable module comprises proprietary technology of the applicant, as embodied in numerous patents and patent applications, it is contemplated that the pressure housing assembly of the present invention will function with differing module or chassis configurations. Thus, although exemplified in the drawings in the present application, other applications of the pressure housing are contemplated and considered within the scope of this invention.

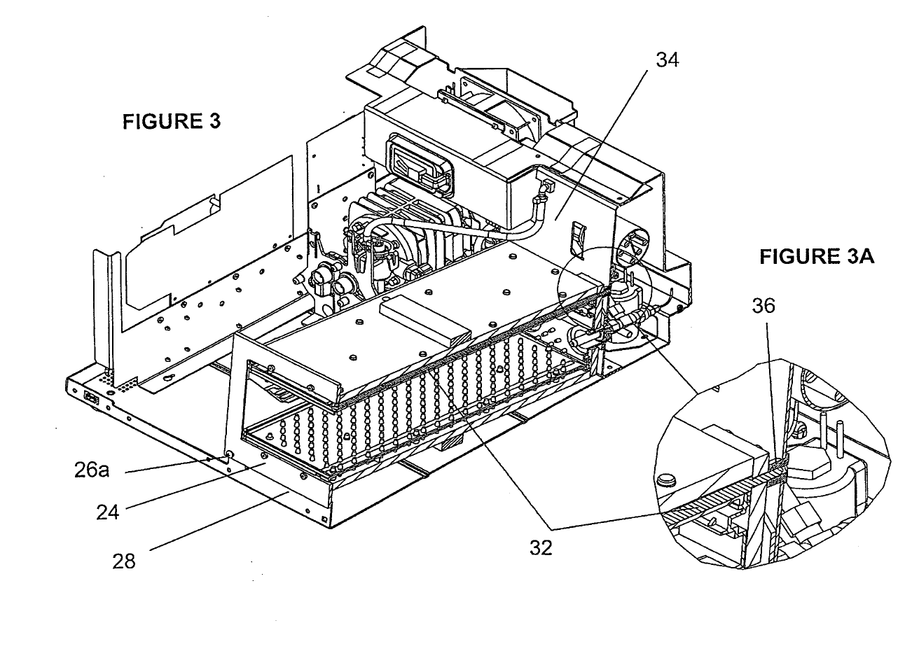

As seen in FIG. 2, the pressure housing 22 attaches to the chassis 20 at 3 points at least. At a front face 24 of the pressure housing 22, there are provided 2 threaded fasteners 26a, 26b that removably fasten to a flange 28 of the chassis 20. The pressure housing 22 is also secured at its back portion to the chassis 20 by means of an extended threaded fastener 32 that runs the length of the pressure housing 22, from its front face 24 to its rear 30. The extended threaded fastener 32 and its configuration and positioning within the pressure housing assembly may be more clearly seen in FIG. 3.

As seen in FIG. 3 and FIG. 3A, in which part of the pressure housing 22 and the apparatus 10 have been cutaway, the extended threaded fastener 32 releasably connects to a chassis bracket 34 on the chassis 20, by means of a threaded bushing 36 that can be permanently attached to the chassis bracket 34. It is also possible to accept the threaded fastener 32 in a tapped aperture or similar configuration that would allow for selective removal of the fastener 32 from the front face 24 of the pressure housing 22.

In this configuration, fasteners 26a, 26b and 32 are shown as screws. However, other similar releasable fasteners, such as partially threaded bolts or lag bolts, releasable using a tool or finger operation or both are contemplated. The extended threaded fastener 32 may be constructed of stainless steel or nickel plated steel, or other suitable materials having the necessary rigidity and strength.

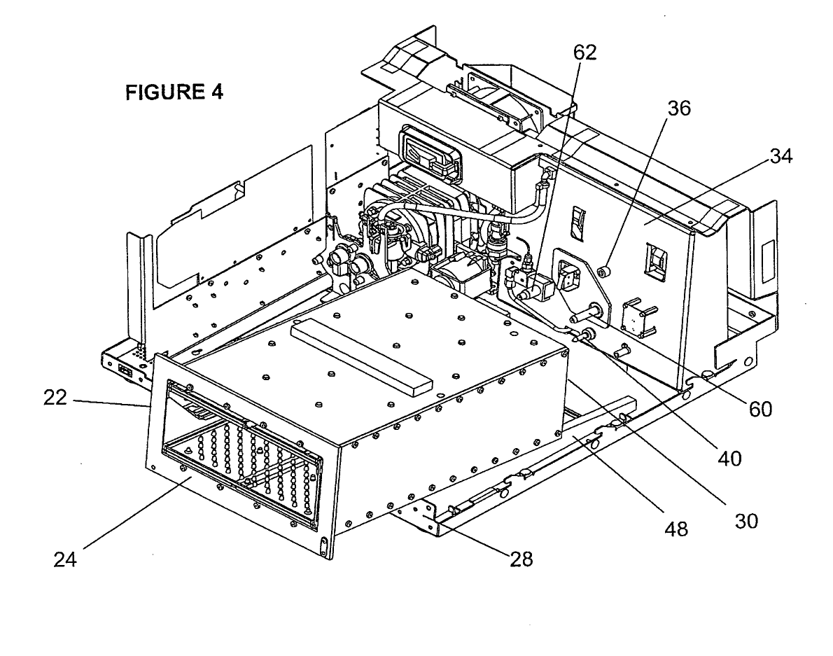

As seen in FIG. 4, once fasteners 26a, 26b and 32 have been removed, or at least disconnected from the chassis flange 28 and bushing 36 respectively, the pressure housing 22 can be removed from operable connection to the chassis 20 and moved forward relative to the chassis 20 for removal from the apparatus 10.

The pressure housing 22 can be similarly easily re-connected to the chassis 20 and placed in operable position within the apparatus 10. The pressure housing 22 must be properly installed and aligned relative to the chassis 20 to ensure that the apparatus is operable and that the cassette 14, when inserted, communicates with the chassis 20 to allow steam to pass through the cassette 14 to sterilize and/or dry the instruments.

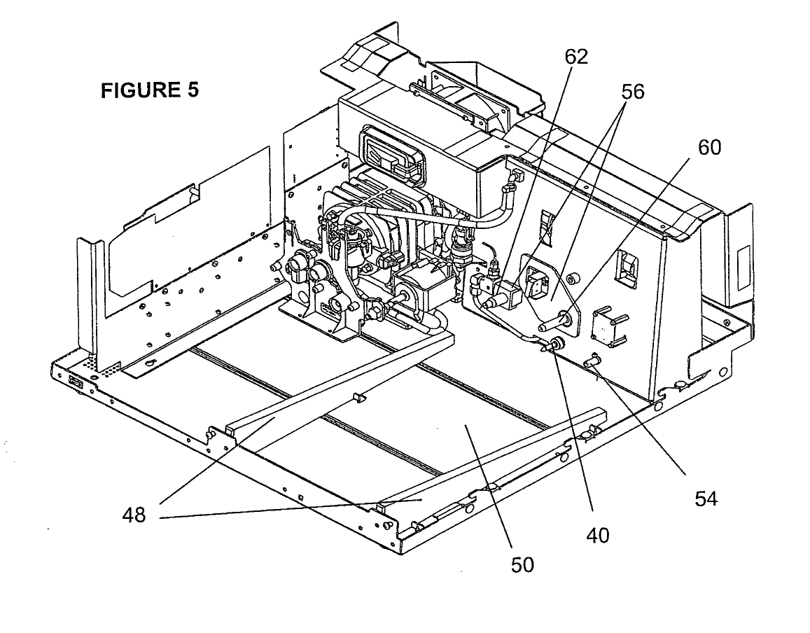

There is thus provided alignment means to guide the re-connection of the pressure housing 22 with the chassis 20. As seen in FIGS. 4 to 6, the alignment means may comprise an alignment pin 40 on the chassis bracket 34 that aligns with alignment aperture 42 on the bracket 44 mounted to the rear face 46 of the pressure housing 22. The alignment means may also comprise guide rails 48 made of Delrin or some other suitable low friction material and mounted on a bottom surface 50 of the chassis 20. These rails 48 help to guide the insertion of the pressure housing 22 to align within the chassis 20 before the fasteners 26a, 26b and 32 are secured. These rails 48 further help to orient the pressure housing 22 at a predefined angle to facilitate condensate drainage in cassette 14.

This also permits a drainage aperture 52 on the pressure housing 22 to align with a drain tube 54 on the chassis 20 to allow any condensate within the pressure housing 22 to drain out to the bottom of the apparatus 10. A flexible silicon tube or the like may be suitable for use as the drain tube 54.

To further prevent any unwanted leakage between the cassette 14 within the pressure housing 22 and the chassis 20, flexible foam gaskets 56 are secured to the chassis bracket 34 to seal against the rear face 46 of the pressure housing 22. This creates a vapour barrier so that any steam leaking from the cassette 14 cannot enter the interior of the serviceable module of the apparatus 10.

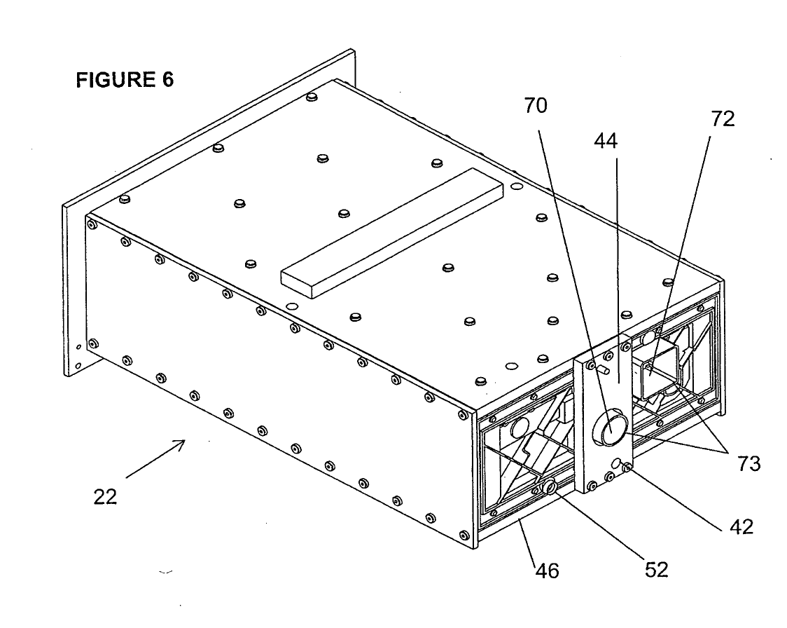

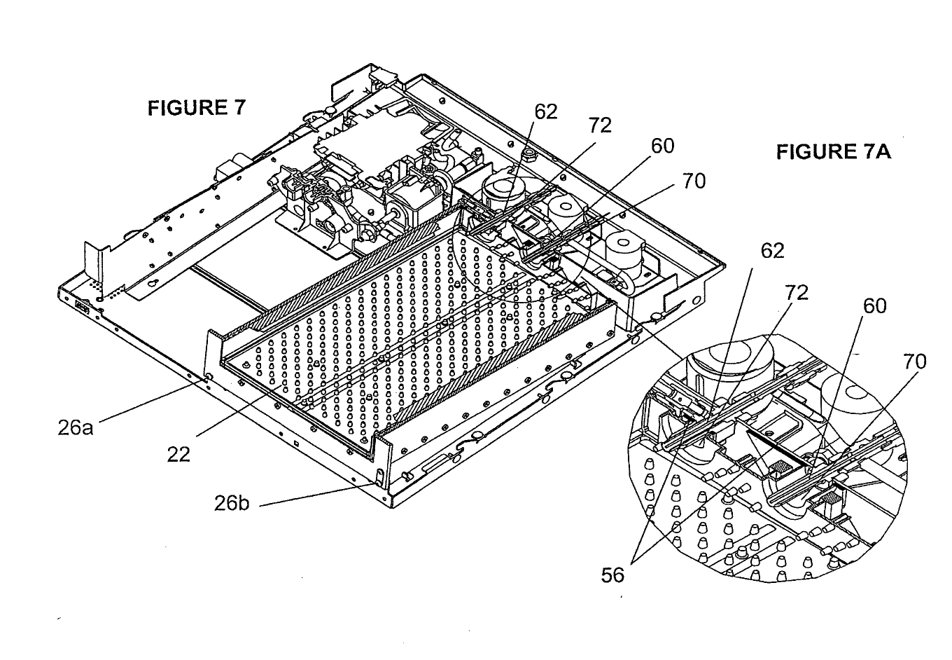

The alignment of the pressure housing 22 within the apparatus 10 is important to allow the passage of steam to the cassette 14 during operation. The steam is passed from the chassis 20 to the cassette 10 by means of a steam inlet probe 60 and exhaust is removed from the cassette 14 by means of the exhaust probe 62 (as seen in FIGS. 7 and 7A). Since the rear face 46 of the pressure housing 22 mates with the chassis bracket 34 in a sealed configuration, the probes 60 and 62 must be allowed to communicate with the cassette 14 via openings in the pressure housing 22. As best seen in FIG. 6, the pressure housing includes at least 2 primary steam openings. The inlet opening 70 on the pressure housing bracket 44 allows the inlet probe 60 to pass through the rear face 46 of the pressure housing 22 to pass steam to the cassette 14, when in position within the pressure housing 22. This also allows a temperature sensor (not shown) housed within the inlet probe 60 to be positioned within the cassette 14 as required. As the temperature sensor is housed within the probe 14, the temperature sensor is protected from potential damage by pressure housing 22 during the insertion of the pressure housing 22 to chassis 20. However, the alignment of cassette 14 after its insertion into pressure housing 22 within the apparatus 10 is still critical to ensure the probe and the temperature sensor are properly placed.

The exhaust opening 72 on the rear face 46 of the pressure housing 22 allows the exhaust probe 62 to pass through the pressure housing 22 into the cassette 14 to allow the egress of steam from the cassette 14. As with probe 60, the exhaust probe 62 can house a temperature sensor that must be placed within the cassette 14 for proper operation. Although the pressure housing 22 is fully decoupled from the plumbing and sensory connections thus allowing the pressure housing 22 to be removably attached to the apparatus 10, the alignment of the cassette 14 upon insertion into the pressure housing 22 with inlet probe 60 and exhaust probe 62 the chassis bracket 34 in the apparatus 10 remains critical.

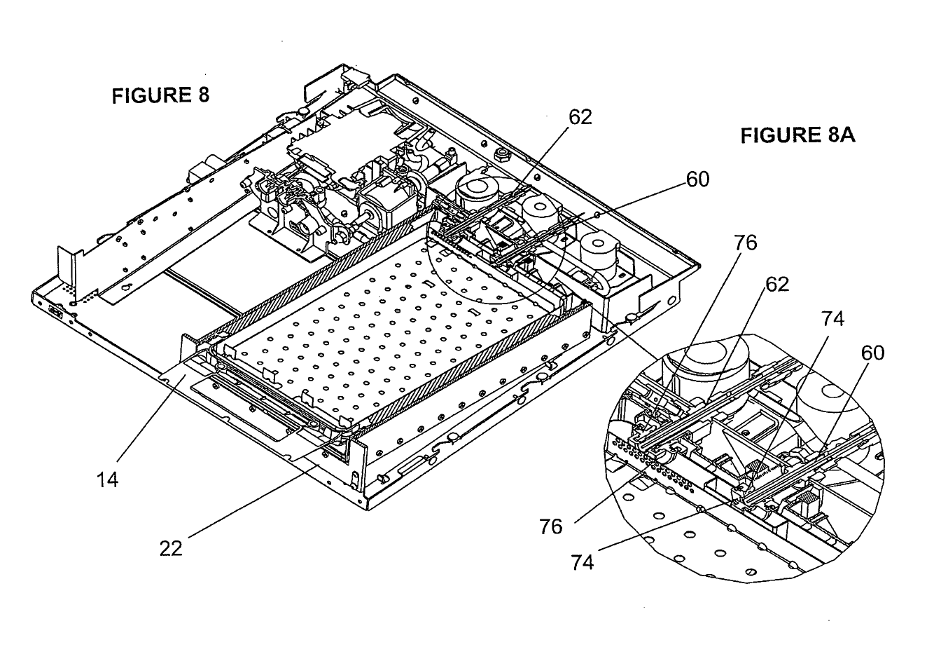

In this regard, in one embodiment of the invention as shown in FIGS. 8 and 8A, the cassette assembly 14 includes a self aligning inlet coupling 74 that aligns with the inlet probe 60 within the inlet opening 70 in order to allow the ingress of steam within the cassette 14. There is also provided a self-aligning exhaust coupling 76 that aligns with the exhaust probe 62 within the exhaust opening 72 to allow the egress of steam from the cassette 14. These self-aligning couplings are described in greater detail in applicant's co-pending international application, Publication No. WO 00/59552.

The gaskets 56 seal against the edges 73 of the openings 70,72 to provide the vapour barrier discussed above.

This type of modular pressure housing 22 thus allows for efficient and convenient coupling and de-coupling of the steam input and exhaust and control functions of the apparatus 10 to the pressure housing 22 and ultimately the cassette 14.

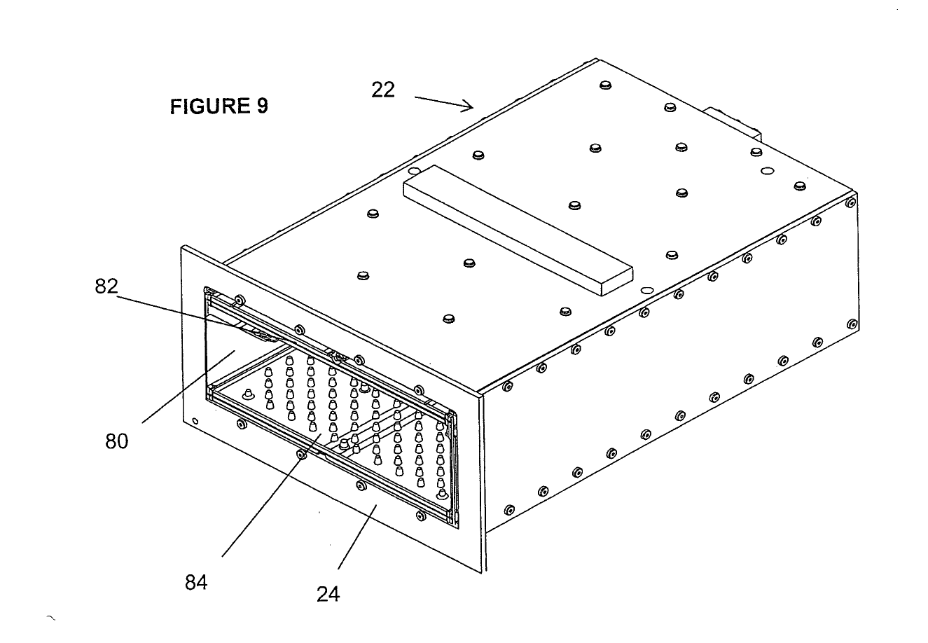

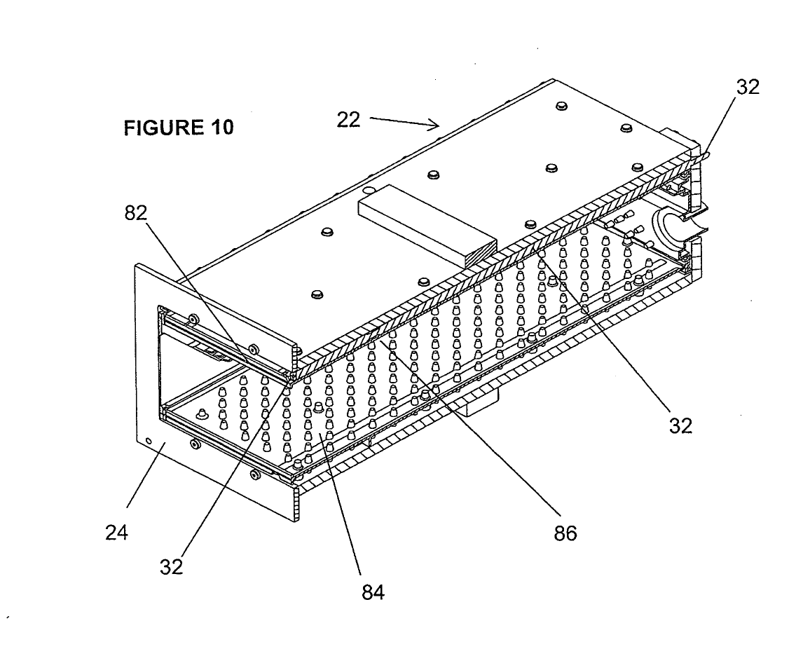

FIGS. 9 and 10 illustrate the pressure housing 22 in perspective view, partially cutaway in FIG. 10, removed from the apparatus 10. The pressure housing 22 is preferably constructed of cold rolled steel, or a combination of cold and hot rolled steel. However, other materials, suitable to withstand the pressure build up within the apparatus due to steam within the cassette 14 and offering rigidity necessary to hold the cassette 14 in place are contemplated. The pressure housing 22 forms a compartment 80 for receiving the cassette 14. The compartment 80 comprises an upper 82 and lower 84 insulation plate to prevent heat loss during operation. A rib 86 in the upper insulation plate 82 is designed to hold the extended threaded fastener 32 in place within the pressure housing 22 to ensure that the extended fastener 32 does not slide completely out of the pressure housing 22 after it is being disconnected from the threaded bushing 36.

A modular pressure housing 22 as described herein would allow a service technician to easily install and service the autoclave apparatus 10 by first removing the pressure housing 22 from the autoclave chassis 20, leaving only the serviceable module, or sterilizing unit to be serviced, or transported for service.

The configuration of the pressure housing and the sterilizing unit thus described would also permit the scalability of cassettes relative to the same sterilizing unit. Using the same configuration of alignment means and openings in a different size of pressure housing will allow a different size of cassette to be inserted into an existing sterilizing unit.

Thus, it is apparent that there has been provided in accordance with the invention a sterilizer with modular pressure housing for a sterilization chamber that fully satisfy the objects, aims and advantages set forth above. While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

1. An apparatus for sterilizing medical or dental instruments comprising:
a sterilizing unit;
a cassette for holding the instruments within said unit during sterilization;
a pressure housing forming a compartment to receive said cassette during use,
wherein said pressure housing releasably connects to said sterilizing unit to allow the ingress and egress of steam into said cassette during sterilization of the instruments and is configured to be easily disconnected, removed from said unit, re-installed and re-connected to said sterilizing unit to allow said cassette to be inserted without any loss of alignment of said cassette relative to said sterilizing unit.

2. The apparatus of claim 1 wherein said sterilizing unit comprises a steam autoclave.

3. The apparatus of claim 1 wherein said sterilizing unit also dries said instruments in said cassette.

4. The apparatus of claim 1 wherein said pressure housing is removably connected to said sterilizing unit by a plurality of mounting means.

5. The apparatus of claim 4 wherein said mounting means comprise at least three threaded fasteners.

6. The apparatus of claim 5 wherein at least one of said threaded fasteners releasably connects to a threaded bushing on said sterilizing unit and at least two of said threaded fasteners releasably connect to threaded apertures on a bottom flange on said sterilizing unit.

7. The apparatus of claim 1 wherein said pressure housing comprises connection means to permit operable connection of said cassette to said sterilizing unit.

8. The apparatus of claim 7 wherein said connection means comprises a first aperture aligned with an inlet probe on said sterilizing unit and a second aperture aligned with an exhaust probe on said sterilizing unit when said pressure housing is attached to said sterilizing unit, allowing said inlet probe and said exhaust probe to communicate the passage of steam to said cassette, when said cassette is in operable position within said pressure housing.

9. The apparatus of claim 1 further comprising an alignment means for aligning said pressure housing with said sterilizing unit.

10. The apparatus of claim 9 wherein said alignment means comprises an alignment pin mounted on a chassis bracket of said sterilizing unit and a corresponding aperture on a rear face of said pressure housing.

11. The apparatus of claim 1 further comprising a drainage means to drain condensate from said pressure housing through said sterilizing unit, when said pressure housing is in position within said sterilizing unit.

12. The apparatus of claim 11 wherein said alignment means further comprises guide rails mounted on a bottom interior face of a chassis of said sterilizing unit for positioning and orienting said pressure housing within said sterilizing unit, wherein said guide rails are downwardly sloped toward a rear of said sterilizing unit, thus aligning said pressure housing at a pre-determined angle within said apparatus, such that said cassette sits within said apparatus at said pre-determined angle, to facilitate draining of condensate from said cassette.

13. An apparatus for sterilizing medical or dental instruments comprising:
a sterilizing unit housing steam delivery means, exhaust means and control means for said apparatus;
a cassette for holding the instruments within said unit during sterilization;
a pressure housing forming a compartment to receive said cassette during use,
wherein said pressure housing is configured to be releasably connectable to said sterilizing unit to permit the efficient coupling and de-coupling of said pressure housing to said steam delivery means, exhaust means and control means.

14. The apparatus of claim 13 wherein said sterilizing unit comprises a steam autoclave.

15. The apparatus of claim 13 wherein said sterilizing unit also dries said instruments in said cassette.

16. The apparatus of claim 13 wherein said pressure housing is removably connected to said sterilizing unit by a plurality of mounting means.

17. The apparatus of claim 13 wherein said mounting means comprise at least three threaded fasteners.

18. The apparatus of claim 17 wherein at least one of said threaded fasteners releasably connects to a threaded bushing on said sterilizing unit and at least two of said threaded fasteners releasably connect to threaded apertures on a bottom flange on said sterilizing unit.

19. The apparatus of claim 13 wherein said pressure housing comprises connection means to permit operable connection of said cassette to said sterilizing unit.

20. The apparatus of claim 19 wherein said connection means comprises a first aperture aligned with said steam delivery means on said sterilizing unit and a second aperture aligned with said exhaust means on said sterilizing unit when said pressure housing is attached to said sterilizing unit, allowing said steam delivery means and said exhaust means to communicate the passage of steam to said cassette, when said cassette is in operable position within said pressure housing.

21. The apparatus of claim 20 wherein said steam delivery means comprises an inlet probe and said exhaust means comprises an exhaust probe.

22. The apparatus of claim 13 further comprising an alignment means for aligning said pressure housing with said sterilizing unit.

23. The apparatus of claim 22 wherein said alignment means comprises an alignment pin mounted on a chassis bracket of said sterilizing unit and a corresponding aperture on a rear face of said pressure housing.

24. The apparatus of claim 23 wherein said alignment means further comprises guide rails mounted on a bottom interior face of a chassis of said sterilizing unit for positioning and orienting said pressure housing within said sterilizing unit, said guide rails sloped downwardly toward a rear of said sterilizing unit, thus aligning said pressure housing at a pre-determined angle within said apparatus, such that said cassette sits within said apparatus at said pre-determined angle, to facilitate draining of condensate from said cassette.
